(19) United States
(12) Patent Application Publication
(10) Pub. No.: US 2018/0344452 A1
(43) Pub. Date: Dec. 6, 2018

(54) Accommodative multifocal intraocular lens

(71) Applicant: VISION PRO (WUXI) LTD, Wuxi (CN)

(72) Inventors: Xiugao LIAO, Wuxi (CN); Zhenyu FENG, Wuxi (CN); Qin YANG, Wuxi (CN)

(73) Assignee: VISION PRO (WUXI) LTD, Wuxi (CN)

(21) Appl. No.: 16/048,354

(22) Filed: Jul. 30, 2018

Related U.S. Application Data
(63) Continuation-in-part of application No. PCT/CN2017/109292, filed on Nov. 3, 2017.

(30) Foreign Application Priority Data
Nov. 16, 2016 (CN) 201611008446.4

(51) Int. Cl. A61F 2/16

(52) U.S. Cl. CPC A61F 2/1618; A61F 2002/1681; A61F 2230/0019; A61F 2250/0091; A61F 2220/0025

(57) ABSTRACT

The present invention is an accommodative multifocal intraocular lens including an optical body, a haptic and a silicone connecting member, wherein the silicone connecting member is located between the optical body and the haptic, and combines the optical body and the haptic in an inserted manner through concave and convex grooves; two ends of the optical body are provided with concave grooves; an end, joined to the optical body, of the haptic is provided with a concave groove; two ends, along the width direction, of the silicone connecting member are provided with convex grooves, respectively, that is, a convex groove and a convex groove, respectively. The optical body in the present invention is a multi-focal optical zone, and the back-and-forth movement of the optical zone can effectively increase the change of the focal power of the multifocal lens.

### CROSS REFERENCE TO RELATED APPLICATIONS

This is a continuation in part application designating US of International Application PCT/CN2017/109292, filed on Nov. 3, 2017, which is based upon and claims priority to Chinese Patent Application No. CN2016110084464, filed on Nov. 16, 2016, the entire contents of which are incorporated herein by reference.

### TECHNICAL FIELD

The present invention relates to the technical field of medical supplies, and in particular to an accommodative multifocal intraocular lens.

### BACKGROUND

The natural lens in the eye of a newborn baby is a colorless, transparency and very soft lens. The ability of the natural lens of newborn baby's eye to change shape and produce dramatic changes in focus is magnificent. As a person ages, the change of external conditions, such as ultraviolet light exposure, the natural lens becomes less clear, more rigid, more color, and functions more like a monofocal lens. When over fifty or sixty years, more than 30 percent of people's crystalline lens will turn yellow or brown or even cloudy. The cloudiness is called a cataract. When this happens, not only lost the accommodative function, but also blurred vision and sensitivity to light. The results will be trouble reading, driving, and less colors vivid. The cataractous lens will have to be replaced with an artificial intraocular lens to restore eyesight of the cataract patient.

A typical intraocular lens is comprised of an optical body and a supporting arm, particularly, the optical body focuses light onto the retina of your eye to enable to see the object. The supporting arm is used to support the optical zone, so that the optical zone of the lens is at the center of the eye which can be focused effectively.

The optical zone and supporting arm of the intraocular lens can be made of the same material or can be made of different materials. The intraocular lens made of the same material is commonly referred to as a one-piece lens, while the intraocular lens made of different materials is commonly referred to as a three-piece lens, and examples thereof are reported in U.S. Pat. No. 4,997,442 and U.S. Pat. No. 5,217,491, among these patents, the optical zones are both made of relatively soft optical materials and arm zone are both made of relatively hard materials.

The conventional monofocal intraocular lens can provide vision correction with a conventional distance, but cannot provide an effective variable-focus vision correction. In other words, it cannot play a role of vision correction both at far and close distances. The only way to make the monofocal intraocular lens work both at far and close distances is to wear a pair of glasses. Another choice is to replace the cataract lens with a multifocal intraocular lens, to make vision at far, close and medium distances available. However, only a part of light at each distance is focused onto the retina of your eye, in addition, multifocal intraocular lens will have some other side effects exist. As a result, people begin to design a novel accommodative intraocular lens, as disclosed in U.S. Pat. No. 4,409,691, U.S. Pat. Nos. 5,674,282, 5,496,366, 6,197,059, and 6,387,126, U.S. Pat. No. 6,178,878, and U.S. Pat. No. 6,406,494. In an accommodating IOL, the haptics are designed to keep the IOL securely in place and prevent any rotational movement, but the legs are flexible in a way that allows the optical portion of the IOL to move slightly forward upon contraction of the ciliary muscle. In this fashion, an accommodating IOL can expand the range of clear vision after cataract surgery, providing better near vision without eyeglasses than what is possible with a conventional monofocal IOL procedure. The optical portion of the IOL to move forward and backward distance is heavily rely on the contraction of the ciliary muscle. For some patients, the contraction of the ciliary muscle is too weak, cannot move the optical body of the intraocular lens, the intraocular lens turn to be the conventional monofocal intraocular lens. And also, the accommodative function will turn to weak along with the increase of the implantation time.

All the designed accommodative intraocular lenses are made of a soft silicone material with a low refractive index. Due to the fact of lower the refractive index of the silicone material, the intraocular lens made from it, is relatively thick, the intraocular lens has a limited distance to move within the capsular bag of your eye, resulted some focusing power back to the ageing eye, but will certainly not restore the same level of focusing available in a young person's eye.

And also, the intraocular lens made from the silicone material will have a higher possibility to form fibers and secondary cataract than the intraocular lens made from hydrophobic polyacrylic ester material.

### SUMMARY

In view of the problems existed in the prior art, the applicant provides an accommodative multifocal intraocular lens design. The optical body of the present invention has a multifocal optical zone, and the back-and-forth movement of the optical zone upon the contraction of the ciliary muscle, can effectively increase the change of the focal power of the multifocal lens. Even if the contraction of the ciliary muscle is too weak, cannot move the optical body of the intraocular lens, the intraocular lens is still turn to be the multifocal intraocular lens.

Technical solutions of the present invention are as follows.

An accommodative multifocal intraocular lens, including an optical body, a haptic and a silicone connecting member, wherein the silicone connecting member is located between the optical body and the haptic, and combines the optical body and the haptic in an inserted manner through concave and convex grooves;
two ends of the optical body are provided with concave grooves; an end, joined to the optical body, of the haptic is provided with a concave groove;
the optical zone of the optical body is an optical zone having two focal points, three focal points, an infinite zoom region or regional multifocal points; and
two ends, along the width direction, of the silicone connecting member are provided with convex grooves, respectively, that is, a convex groove and a convex groove, respectively.

The concave and convex grooves are rectangular or dovetail-shaped.

The intraocular lens is 11.5 to 13.5 mm in diameter.

The optical body is made of an optically transparent hydrophobic polyacrylic ester material having a refractive index of 1.48 to 1.56; the effective optical zone of the optical body is 5.5 to 6.5 mm in diameter.

The haptic is made of hydrophobic polyacrylic ester material or reinforced silicone material, and the haptic is 0.18 to 0.65 mm in thickness; the thickest portion of the silicone connecting member is 0.18 to 0.65 mm in thickness.

The concave groove and the concave groove are both 0.1 to 0.15 mm in depth; the convex groove and the convex groove are both 0.1 to 0.15 mm in height; the depths of the concave groove and the groove are consistent with the heights of the convex groove and the convex groove.

The widths of the convex groove and the convex groove are consistent with the widths of the concave groove and the concave groove.

An accommodative multifocal intraocular lens, including an optical body, a haptic and a silicone connecting member, wherein the silicone connecting member is located between the optical body and the haptic, and combines the optical body and the haptic in an inserted manner through concave and convex grooves;

convex grooves are provided on a cross section along the thickness direction, at two ends of the optical body; a convex groove is provided on a cross section along the thickness direction at an end, joined to the optical body, of the haptic; and
concave grooves are provided on a cross section along the thickness direction, at two ends, along the width direction of the silicone connecting member.

The material in the optical zone of the present invention is an optically transparent hydrophobic polyacrylic ester material, the material also can be acrylate, methacrylate or styrene derivative and small amounts of hydrophilic monomers, such as 2-hydroxyethyl methacrylate, 2-hydroxyethyl acrylate, 3-hydroxypropyl methacrylate, 3-hydroxypropyl acrylate, 4-hydroxybutyl methacrylate, 4-hydroxybutyl acrylate, N,N-dimethylacrylamide, N,N-dimethymethacrylamide, N,N-diethylacrylamide, N-ethylacrylamide.

The accommodative multifocal intraocular lens can first be made into an intraocular lens disk containing a multifocal optical surface, mechanically engraved into a one-piece lens, and then cut into an optical body and two haptics, and silicone connecting member is made by laminate molding.

The beneficial effects of the present invention lie in that: the silicone connecting member adopted in the present invention is a relatively soft material with good elasticity, when intraocular muscles contract and relax, the intraocular optical zone can be effectively moved back and forth.

The optical zone of the optical body in the present invention is an optical zone having two focal points, three focal points, an infinite zoom region or regional multifocal points; when the contraction or relaxation of the ciliary muscle, the original bifocal, trifocal, an infinite zoom region, or regional multifocal lens is changed into a new bifocal, trifocal, an infinite zoom region, or regional multifocal lens, and the optical power range will increase change in the optical power. Since the optical zone of the multifocal lens moves back and forth when the contraction of the ciliary muscle, the original bifocal, trifocal, an infinite zoom region, or regional multifocal lens is changed into a new bifocal, trifocal, an infinite zoom region, or regional multifocal lens, and the optical power range will increase change in the optical power. As such, the accommodative optical power if the newly designed accommodative multifocal intraocular lens will be greater than the optical power range of any accommodative multifocal intraocular lens available in the market. Even if the lens loses the back-and-forth movement function for several reasons after several years, the lens will still maintain the original bifocal, trifocal, an infinite zoom region, or regional multifocal function.

When the accommodative multifocal intraocular lens moves back and forth, moving distances and optical power changes are as shown in table below:

| Moving | Diopter range | | | | | |
|---|---|---|---|---|---|---|
| distance | 10.0-13.5 D | | 20.0-23.5 D | | 30.0-33.5 D | |
| 0.0 mm | 10.0 D | 13.5 D | 20.0 D | 23.5 D | 30.0 D | 33.5 D |
| 0.25 mm | 10.34 D | 13.84 D | 20.34 D | 23.84 D | 30.34 D | 33.84 D |
| 0.50 mm | 10.70 D | 14.20 D | 20.70 D | 24.20 D | 30.70 D | 34.20 D |
| 0.75 mm | 11.05 D | 14.55 D | 21.05 D | 24.55 D | 31.05 D | 34.55 D |
| 1.00 mm | 11.42 D | 14.92 D | 21.42 D | 24.92 D | 31.42 D | 34.92 D |
| 1.25 mm | 11.81 D | 15.31 D | 21.81 D | 25.31 D | 31.81 D | 35.31 D |
| 1.50 mm | 12.24 D | 15.74 D | 22.24 D | 25.74 D | 32.24 D | 35.74 D |
| 1.75 mm | 12.67 D | 16.17 D | 22.67 D | 26.17 D | 32.67 D | 36.17 D |
| 2.00 mm | 13.11 D | 16.61 D | 23.11 D | 26.61 D | 33.11 D | 36.61 D |

### BRIEF DESCRIPTION OF THE DRAWINGS

FIG. 1 is a schematic structural diagram according to the present invention;

FIG. 2 is a schematic structural diagram of an optical body according to the present invention;

FIG. 3 is a perspective schematic structural diagram of an optical body according to the present invention;

FIG. 4 is a schematic structural diagram of a haptic according to the present invention;

FIG. 5 is a perspective schematic structural diagram of a haptic according to the present invention;

FIG. 6 is a schematic structural diagram of a silicone connecting member according to the present invention;

FIG. 7 is a perspective schematic structural diagram of a silicone connecting member according to the present invention; and

FIG. 8 is a schematic structural diagram according to the present invention.

In the figures: 1. optical body, 2. haptic, 3. silicone connecting member, 4. concave groove, 5. concave groove, 6. convex groove, 7. convex groove, 8. convex groove, 9. convex groove, 10. concave groove.

### DETAILED DESCRIPTION OF THE EMBODIMENTS

The present invention will be described in detail in conjunction with the accompanying drawings and embodiments.

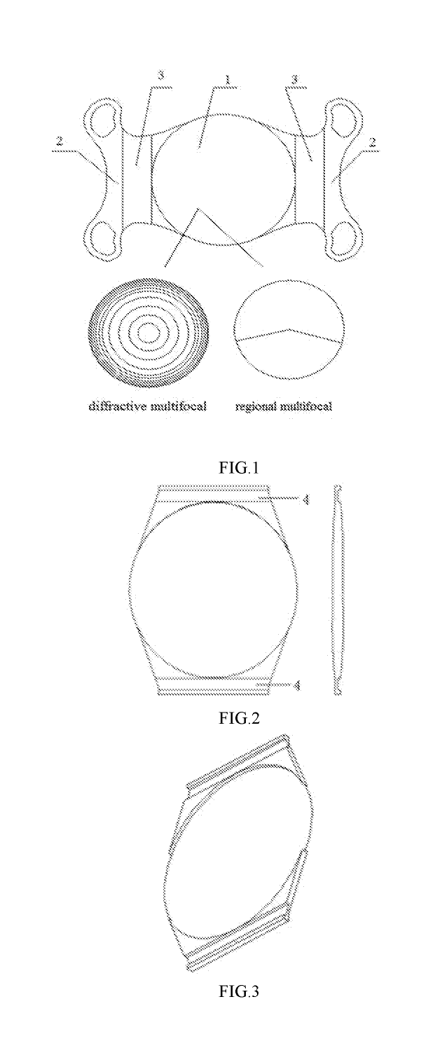

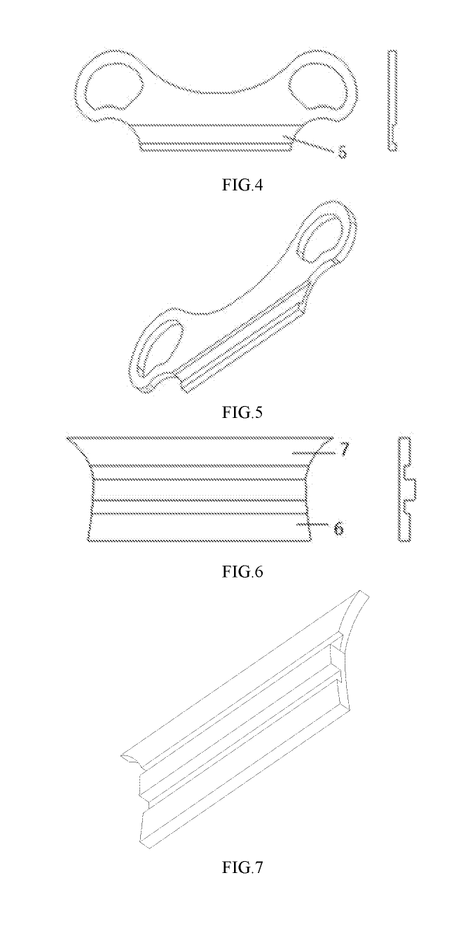

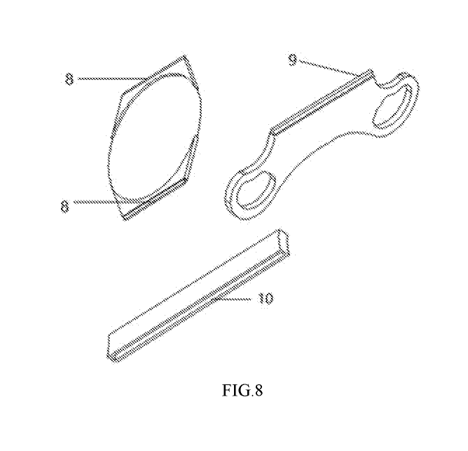

As shown in FIG. 1 to FIG. 7, an accommodative multifocal intraocular lens, including an optical body 1, a haptic 2 and a silicone connecting member 3, wherein the silicone connecting member 3 is located between the optical body 1 and the haptic 2, and combines the optical body 1 and the haptic 2 in an inserted manner through concave and convex grooves; two ends of the optical body 1 are provided with concave grooves 4; an end, joined to the optical body 1, of the haptic 2 is provided with a concave groove 5; the optical zone of the optical body 1 is an optical zone having two focal points, three focal points, an infinite zoom region or regional multifocal points;

two ends, along the width direction, of the silicone connecting member 3 are provided with convex grooves, respectively, that is, a convex groove 6 and a convex groove 7, respectively.

The concave and convex grooves are rectangular or dovetail-shaped.

The intraocular lens is 11.5 to 13.5 mm in diameter.

The optical body 1 is made of an optically transparent hydrophobic polyacrylic ester material having a refractive index of 1.48 to 1.56; the effective optical zone of the optical body 1 is 5.5 to 6.5 mm in diameter.

The haptic 2 is made of hydrophobic polyacrylic ester material or reinforced silicone material, and the haptic 2 is 0.18 to 0.65 mm in thickness; the thickest portion of the silicone connecting member 3 is 0.18 to 0.65 mm in thickness.

The concave groove 4 and the concave groove 5 are both 0.1 to 0.15 mm in depth; the convex groove 6 and the convex groove 7 are both 0.1 to 0.15 mm in height; the depths of the concave groove 4 and the groove 5 are consistent with the heights of the convex groove 6 and the convex groove 7.

The widths of the convex groove 6 and the convex groove 7 are consistent with the widths of the concave groove 4 and the concave groove 5.

When in use, convex and concave grooves of two silicone connecting members 3 are applied with medical strong glue first, loaded on two ends of the optical body 1, and the convex grooves at two ends of the optical body 1 are inserted into the concave grooves of the silicone connecting member 3, and correspondingly, concave grooves at two ends of the optical body 1 coincide with the convex grooves of the silicone connecting member 3, and then the convex grooves at the other end of the two silicone connecting members 3 are loaded in the concave grooves of the two haptics 2, as such, the overall assembly of the three components is completed, and finally implanted into the capsular bag of the human eye.

As shown in FIG. 8, an accommodative multifocal intraocular lens, including an optical body 1, a haptic 2 and a silicone connecting member 3, wherein the silicone connecting member 3 is located between the optical body 1 and the haptic 2, and combines the optical body 1 and the haptic 2 in an inserted manner through concave and convex grooves;

convex grooves 8 are provided on a cross section along the thickness direction, at two ends of the optical body 1; a convex groove 9 is provided on a cross section along the thickness direction at an end, joined to the optical body 1, of the haptic 2; and
concave grooves 10 are provided on a cross section along the thickness direction, at two ends, along the width direction of the silicone connecting member 3.

When in use, convex grooves 8 and 9 are applied with medical strong glue first, and then inserted into the concave grooves 10 on a cross section along the thickness direction, at two ends, along the width direction of the silicone connecting member 3, respectively, as such, the overall assembly of the three components is completed, and finally implanted into the capsular bag of the human eye.

### Embodiment 1

Referring to FIG. 1 to FIG. 7, an accommodative trifocal intraocular lens is an assembled intraocular lens with three focal points of 13.5 D, 20.0 D, 23.5 D, respectively, and the preparation method thereof is:

(1) an intraocular lens disk (13 mm in diameter) containing a trifocal optical surface can first be made;
(2) the disk made in step (1) is mechanically engraved into a one-piece lens, and then cut into an optical body and two haptics, and silicone connecting member is made by laminate molding;
(3) convex and concave grooves of two silicone connecting members are applied with medical strong glue first, loaded on two ends of the optical body, and the convex grooves at two ends of the optical body are inserted into the concave grooves of the silicone connecting member, and correspondingly, concave grooves at two ends of the optical body coincide with the convex grooves of the silicone connecting member, and then the convex grooves at the other end of the two silicone connecting members are loaded in the concave grooves of the two haptics, as such, the overall assembly of the three components is completed, and finally implanted into the capsular bag of the human eye;
the optical body is made of a hydrophobic polyacrylic ester material and the effective optical zone of the optical body is 5.5 mm in diameter and 0.6±0.2 mm in thickness;
the haptic is made of hydrophobic polyacrylic ester material and the haptic is 0.3±0.1 mm in thickness;
the overall width of the silicone connecting member is 1.5 mm and the thickness in the center is 0.3 mm; the convex groove in the middle of the silicone connecting member is 0.3 mm in width, convex grooves at two ends are both 0.4 mm in width, and concave grooves in the middle are both 0.2 mm in width.

When the contraction or relaxation of the ciliary muscle, the back-and-forth moving distance is 1.5 mm, and the zoom regions of the intraocular lens in the present invention are in ranges of 13.5 D-15.74 D, 20.0 D-22.24 D and 23.5 D-25.74 D.

### Embodiment 2

Referring to FIG. 1 to FIG. 7, an accommodative trifocal intraocular lens is an assembled intraocular lens with three focal points of 10.0 D, 20.0 D, 30.0 D, respectively, and the preparation method thereof is:

(1) an intraocular lens disk (13 mm in diameter) containing a trifocal optical surface can first be made;
(2) the disk made in step (1) is mechanically engraved into a one-piece lens, and then cut into an optical body and two haptics, and silicone connecting member is made by laminate molding;
(3) convex and concave grooves of two silicone connecting members are applied with medical strong glue first, loaded on two ends of the optical body, and the convex grooves at two ends of the optical body are inserted into the concave grooves of the silicone connecting member, and correspondingly, concave grooves at two ends of the optical body coincide with the convex grooves of the silicone connecting member, and then the convex grooves at the other end of the two silicone connecting members are loaded in the concave grooves of the two haptics, as such, the overall assembly of the three components is completed, and finally implanted into the capsular bag of the human eye;
the optical body is made of a hydrophobic polyacrylic ester material and the effective optical zone of the optical body is 6 mm in diameter and 0.6±0.2 mm in thickness;
the haptic is made of hydrophobic polyacrylic ester material and the haptic is 0.3±0.1 mm in thickness;
the overall width of the silicone connecting member is 1.2 mm and the thickness in the center is 0.3 mm; the convex groove in the middle of the silicone connecting member is 0.1 mm in width, convex grooves at two ends are both 0.4 mm in width, and concave grooves in the middle are both 0.15 mm in width.

When the contraction or relaxation of the ciliary muscle, the back-and-forth moving distance is 1.25 mm, and the zoom regions of the intraocular lens are in ranges of 10.0 D-11.81 D, 20.0 D-21.81 D and 30.0 D-31.81 D.

### Embodiment 3

Referring to FIG. 8, an accommodative trifocal intraocular lens is an assembled intraocular lens with three focal points of 10.0 D, 20.0 D, 30.0 D, respectively, and the preparation method thereof is:

(1) an intraocular lens disk (13 mm in diameter) containing a multifocal optical surface can first be made;
(2) the disk made in step (1) is mechanically engraved into a one-piece lens, and then cut into an optical body and two haptics, and silicone connecting member is made by laminate molding;
(3) convex grooves at two ends of the optical body and convex grooves of the two haptics are applied with medical glue, and then inserted into the concave grooves at two ends of the silicone connecting member, as such, the overall assembly of the three components is completed, and finally implanted into the capsular bag of the human eye;
the optical body is made of a hydrophobic polyacrylic ester material and the effective optical zone of the optical body is 6 mm in diameter and 0.6±0.2 mm in thickness;
the haptic is made of hydrophobic polyacrylic ester material and the haptic is 0.3±0.1 mm in thickness;
the overall width of the silicone connecting member is 0.4 mm and the thickness in the center is 0.2 mm and the width in the center is 0.1 mm; concave grooves are provided at two ends of silicone connecting member, and are both 0.15 mm in width and 0.1 mm in thickness, bosses are provided at two ends of the concave groove, so as to achieve insertion and assembly of the body and the haptic.

When the contraction or relaxation of the ciliary muscle, the back-and-forth moving distance is 1.25 mm, and the zoom regions of the intraocular lens are in ranges of 10.0 D-11.81 D, 20.0 D-21.81 D and 30.0 D-31.81 D.

What is claimed is:

1. An accommodative multifocal intraocular lens comprising:
an optical body, a haptic, and a silicone connecting member; wherein
the silicone connecting member is located between the optical body and the haptic, and
the silicone connecting member combines the optical body and the haptic in an inserted manner through concave and convex grooves;
two ends of the optical body are both provided with a first concave groove;
an end, joined to the optical body, of the haptic is provided with a second concave groove;
an optical zone of the optical body is an optical zone having two focal points, three focal points, an infinite zoom region, or regional multifocal points; and
two ends, along a width direction, of the silicone connecting member are both provided with the convex groove, respectively, the convex groove include a first convex groove and a second convex groove.

2. The accommodative multifocal intraocular lens according to claim 1, wherein
the concave and convex grooves are rectangular or dovetail-shaped.

3. The accommodative multifocal intraocular lens according to claim 1, wherein
a diameter of the intraocular lens ranges from 11.5 mm to 13.5 mm.

4. The accommodative multifocal intraocular lens according to claim 1, wherein
the optical body is made of an optically transparent hydrophobic polyacrylic ester material having a refractive index ranges from 1.48 to 1.56; and
a diameter of an effective optical zone of the optical body ranges from 5.5 mm to 6.5 mm.

5. The accommodative multifocal intraocular lens according to claim 1, wherein
the haptic is made of a hydrophobic polyacrylic ester material or a reinforced silicone material, and a thickness of the haptic ranges from 0.18 mm to 0.65 mm; and
a thickness of a thickest portion of the silicone connecting member ranges from 0.18 mm to 0.65 mm.

6. The accommodative multifocal intraocular lens according to claim 1, wherein,
a depth of the first concave groove and the second concave groove ranges from 0.1 mm to 0.15 mm;
a height of the first convex groove and the second convex groove ranges from 0.1 mm to 0.15 mm; and
the depth of the first concave groove and the second groove is consistent with the height of the first convex groove and the second convex groove.

7. The accommodative multifocal intraocular lens according to claim 1, wherein
a width of the first convex groove and the second convex groove is consistent with a width of the first concave groove and the second concave groove.

8. An accommodative multifocal intraocular lens, comprising:
an optical body, a haptic, and a silicone connecting member; wherein
the silicone connecting member is located between the optical body and the haptic, and combines the optical body and the haptic in an inserted manner through concave and convex grooves;
third convex grooves are provided on a cross section along a thickness direction, at two ends of the optical body;
a fourth convex groove is provided on a cross section along a thickness direction at an end, joined to the optical body, of the haptic; and
third concave grooves are provided on a cross section along a thickness direction, at two ends, along a width direction of the silicone connecting member.
